(19) United States
(12) Patent Application Publication
(10) Pub. No.: US 2008/0166395 A1
(43) Pub. Date: Jul. 10, 2008

(54) ARTICLE FOR DELIVERING SUBSTRATES TO THE ORAL CAVITY

(76) Inventor: Steven H. Roush, Sarasota, FL (US)

Correspondence Address: ROBERT M. SCHWARTZ, P.A., P.O. BOX 221470, HOLLYWOOD, FL, 33022, US

(21) Appl. No.: 11/968,910

(22) Filed: Jan. 3, 2008

Related U.S. Application Data
(60) Provisional application No. 60/883,438, filed on Jan. 4, 2007.

(51) Int. Cl. A61K 9/00; A61K 31/4439; A61P 25/34

(52) U.S. Cl. 424/439; 514/343; 424/400

(57) ABSTRACT

The present invention is an oral dissolvable pouch with a substrate, for providing therapy and a method of providing therapy using the pouch of the present invention.

### INDEX TO RELATED APPLICATIONS

This application claims benefit of U.S. Provisional Patent Application Ser. No. 60/883,438 filed Jan. 4, 2007 the disclosure of which is incorporated herein by reference in its entirety.

### BACKGROUND OF THE INVENTION

Smokeless tobacco pouches for chewing have been popular since around the 1980s. A typical pouch is made of a fine cloth or mesh type material that holds the tobacco completely enclosed in an interior cavity, but lets the juices permeate through the pouch walls. The pouch is not edible, does not dissolve, and is eventually discarded from the mouth.

There are various products on the market purporting similar aspects of the subject invention.

Oregon Mint Snuff Company filled a pouch that is not edible or dissolvable with non-tobacco ingredients (crystallized mint, other ingredients).

Nicotine Replacement Therapies (NRT)—Nicotine gum is recommended to be chewed a couple of times and is then placed between the cheek and gum to deliver nicotine. Many people don't like chewing gum and it doesn't “park” as well as tobacco or coconut or other herbal alternatives.

KIKIT Corporation of Sarasota Fla. has coconut in a can similar to a smokeless tobacco can. Liquid ingredients are added to the dry coconut to give it moisture, flavor and preserve for shelf stability. The Kikit products have unique blend of ingredients. Nicotine, herbs, vitamins, etc. have not yet been added to the coconut base. However, the coconut does serve as a base ingredient to act as a delivery vehicle of juices and flavors. Loose, moist coconut is placed between cheek and gum. Users can then nibble on the pieces or eat it or discard it.

Other attempts have been made using herbal and other tobacco chew alternative products. Base ingredients used include: Tealeaves, mint leaves, clover, tobacco stems instead of leaves, ginseng, shredded beef jerky, grape leaves. Most of these are loose and in a can like a round tobacco can. Some are available in pouches, but none in an edible/dissolvable pouch.

Having a food based product that has nicotine in it will allow the user to initially get a measured dose of nicotine without the harmful effects of the carcinogens found in tobacco. For smokeless tobacco users, the use of the pouch would satisfy a habit very similar to what they are accustomed to doing.

Blue Whale is made of tealeaves and also contains a botanical derivative of nicotine called NicoDip™.

Mint Snuff Pouches are miniature tea bags filled with mint and crystallized mint oil or other flavors.

Nicotine Replacement Therapy (NRT) Gums, Nasal Sprays, Inhalers, Patches, and Pills have become very important in helping reduce tobacco usage.

There is currently not a pouch product used for NRT. A pouch more closely resembles texture and feel of chewing tobacco. The present invention is an edible Pouch with outer liner that could be a paper type liner that has the hand and mouth “feel” of tobacco paper as used in cigarettes and cigars. An advantage of the pouches in the present invention would have over herbal pouch products or leaf products or certainly tobacco products if put in edible pouches is that the consumer would consider more “edible” than the other options (tobacco, ground up tea or mint or other leaves). Ginseng (and other like products . . . Ginkgo) or shredded beef jerky however would be edible and could be put in an edible pouch.

There is a need for a pouch product to provide therapy in general and nicotine replacement therapy in particular.

### BRIEF SUMMARY OF THE INVENTION

The present invention is an article for delivering a product to the oral cavity having at least one dissolvable material used as a liner, with the liner forming a pouch that completely encloses an inner cavity and a substrate placed within the inner cavity.

In one embodiment, the pouch is formed of a material with a single dissolution rate. Alternatively, the article may be formed of at least two dissolvable materials that have different rates of dissolution in the oral cavity.

The pouch may provide medicinal or nutritional therapy to a user.

In a preferred embodiment, the article pouch has a quantity of nicotine suitable for nicotine replacement therapy. Although suitable amounts vary, the present invention is contemplated to provide up to 21 mg nicotine per dose. The therapy may be up to 60 mg nicotine or more depending on therapy desired. Additionally, the pouches may contain varying amounts of nicotine such that as therapy progresses, decreasing amounts of nicotine may be delivered.

In one embodiment, the therapeutic agent, whether medicinal or nutritional may be incorporated into the substrate contained within the pouch, the material comprising the body of the pouch, or a combination thereof.

The article has a substrate comprising a base material that is an edible substance. A preferred base material is coconut.

The substrate may also comprise any other desired components including, but not limited to, flavor, color, pharmaceuticals, nutritional supplements and the like incorporated into said article.

The therapeutic agent may be incorporated into the lining of the pouch, one or more liners, and/or the inner substrate.

Also contemplated is a method for providing nicotine replacement therapy having the steps of producing articles described herein that incorporate therapeutic amounts of nicotine for replacement therapy and administering to a person said articles with nicotine content decreasing over time.

The method need not be limited to providing nicotine therapy, but may provide any medicinal or nutritional therapy.

There are many available materials for forming articles of the present invention.

Watson http://www.watson-inc.com/ has films that dissolve and are edible. An example of how used is Listerine Pocket Packs—Oral Care Strips (Pfizer).

Watson also sells bakery ingredients that are available in dissolvable pouches (actually “bags” vs. small pouches. The bags contain a measured amount of ingredients. A baker places a certain quantity of bags in a batch. The bag dissolves away allowing ingredients to be freed. The bag liner material is a thin paper type material. It dissolves more slowly than films like used in Listerine type strips mentioned above.

There are numerous materials available, in addition to those available from Watson, to make a dissolvable pouch for the present invention.

Dissolvo® is a dissolvable paper available from DayMark Safety Systems of Bowling Green, Ohio.

Additionally, dissolvable surgical stitch material, comprising collagen and the like, may be used to form dissolvable pouches.

The present invention may be a “Recreational Chew Alternative”—and encompass an energy chew, breath freshener, alternative to gum, breath mints, sunflower seeds, and beef jerky.

In one embodiment, the present invention may be a tobacco Cessation tool and/or tobacco alternative for chewers (smokeless tobacco) or smokers (cigar, pipe, and cigarette), The present invention may also be a medicinal—Insulin, Vitamins, Herbs, Oral Medicines, i.e. deliver something that helps improve gums. (oral therapy for gingivitis, gum disease, etc.).

The present invention is advantageous because the pouch and/or contents may be edible and/or dissolvable.

The invention provides for the use of an edible substrate, such as coconut (Herbs like Ginseng and Shredded Beef Jerky etc) as a base ingredient for completely edible product contained within an edible and/or dissolvable pouch. Dry (desiccated) or moist coconut is combined with dry or liquid ingredients for oral fixation satisfaction, psychological, physiological and/or medicinal and other purposes. Coconut is loose or may be encased in edible and/or dissolvable pouches, tubes or could be in non-edible pouches like are currently used.

The ritual of taking a pinch of coconut or a pouch with a pinch of coconut in it and placing it in the mouth is a way to satisfy a habit that does not contain the unhealthy carcinogens known to be in tobacco (smoking or chewing) products; does not stain teeth like tobacco or other leaf (i.e. mint, tea, grape) type products; is commonly considered more edible than tobacco or other leaf (i.e. mint, tea, grape) type products, or plant parts, clover or corn silk.

Coconut may also serve as an alternative to other products such as chewing gum, breath mints, candy, sunflower seeds, toothpicks, beef jerky (or shredded beef jerky), ginseng, that might not be desirable alternatives to those seeking to satisfy an oral fixation and/or hand to mouth ritual, halitosis, cleaner mouth feel, or for other social reasons. Shredded beef jerky is similar in that it is a “food” alternative like coconut, but to the user, it dissolves more than coconut and will typically not last as long. Ginseng is another food alternative that is used as a tobacco alternative. It pinches and packs similarly, but is more slippery and does not stay packed between cheek and gum as well as coconut. Also, problems with above such as sunflower seed shells, toothpick is when user is finished using, and they must be discarded because they are not edible.

Dissolvable and/or edible paper (i.e. as used for bags (pouches) of ingredients in the baking industry) or dissolvable and/or edible film (i.e. like Listerine Pocket Packs) makes up the pouch or tubes outside liner (note: could have both films on outside laminated with more of a paper on the inside). Purpose would be to have moist feel, flavor burst etc that touches lining of the mouth initially with a more protective drier inside paper type material that touches dry ingredients inside or to form a barrier for moist ingredients inside.

The coconut or film or paper liners or a combination of these as used in pouches/tubes may serve as a base to which therapeutic agents may be delivered to a person in need of therapy. Said therapeutic may include, but would not be limited to pharmaceuticals, nutritional supplements such as nicotine, caffeine, herbs, vitamins, medicine (insulin), may be added and that when placed between the cheek and gum in the mouth will deliver a measured dose for relaxation, energy (alertness—people, i.e. students, military personnel, truck drivers say they chew to stay awake), medicinal or other physiological purposes.

The coconut or film or paper liners or a combination of these as used in pouches/tubes may be aroma enhanced, flavored, or colored and may contain natural and/or artificial preservatives.

If white coconut is used a preferred preservative is sodium metabisulfite.

The coconut may be one or a combination of the many available cuts of coconut. One wholesale supplier offers ten different cuts of coconut in a dry desiccated form and also has sweetened and also has toasted varieties. The sweetened and toasted coconut uses one or more of the ten cuts. Most cuts are sulfured, which is considered a preservative even though it occurs naturally in coconut. The sulfur keeps it white. Since the product would be in a pouch, it would not need to stay as white as if visually exposed, so in one embodiment, an unsulphured coconut may be used. Regarding cuts . . . most likely it will be one or a combination of the two or three finest cuts: Extra Fine, Fine (or sometimes called Macaroon) Medium.

Similar cuts may be available and used with such articles as Ginseng cuts or beef/poultry. A preferred embodiment is coconut as a base. For filling such a small pouch it is contemplated to use an extra fine or fine cut or a combination of the two.

Fresh breath film like “Listerine Pocket Packs” serve as breath freshener or flavor burst, but do not have substance for lasting effect as is gained with flavored coconut or coconut used in combination with film or paper as in pouches/tubes.

Coconut as the base ingredient inside the pouch provides a substance that will park well between the cheek and gum or under the tongue without sliding around like other known food type ingredients. The coconut is also absorbent, so as the dry ingredients take on moisture from the dissolving liner and saliva of the mouth, the coconut will moisten. The coconut will then be appealing as something parked between the cheek and gum, and/or nibble on and/or eat.

Using a pouch in combination with the coconut allows for better dose control of nicotine, (i.e. nicotine salt mixed with the dry coconut) as compared to loose coconut. Rather than the user taking a pinch of varying sizes (depending on their hand/finger size or just how much they pinch or grab) of loose coconut/nicotine, there will be a measurable amount in the pouch. For example, a pouch may contain for example 1, 3, 7, 10, 15, 21, or more mg of nicotine per pouch.

A second liner with nicotine is also a possibility. This second, inner liner would be laminated or folded into the outer liner. The liner or liners may also possibly have nicotine in it.

The nicotine could be delivered in several ways. For example:

A. Nicotine salt added to dry coconut blend. User only gets to nicotine once the outer liner(s) dissolves.
B. Inside liner only has nicotine in it. It would dissolve more slowly over time than say the
thinner outside liner (if used). The more paper like inner liner could also “be” the outer liner. It would have more of a paper mouth feel, more similar to cigarette paper.

Outside liner (or only liner) could be a thin film, like is used with Listerine Pocket Packs Oral Care Strips that could deliver nicotine. This would be a nicotine “burst” that would not last very long, but could quickly calm the user.

An advantage of this invention over strips is contents of pouch would last awhile and give user something to satisfy the longer term oral fixation they are accustom to from smoking, chewing tobacco, mints, gum, toothpicks, sunflower seeds, etc.

A combination of any or all of the options mentioned above.

In a preferred embodiment, the article is to be placed on the outer portion of the teeth next to the interior portion of the cheek.

There are essentially two parts to dealing with tobacco cessation. One is the physical cravings due to nicotine withdrawal and the other has to do with the habit and/or social aspects. This invention, unlike most other nicotine replacement therapy products, addresses both for both the smoker and the smokeless tobacco user.

The nicotine inhaler and nicotine gum both attempt to address both to some degree. The other methods, the patch, nasal spray and capsules do not address both. They address the nicotine withdrawals, but not the habit.

The present invention preferably contains coconut (or other edible substrate) in a dissolvable pouch, is more similar to the habit specifically for smokeless tobacco users in how the product is taken and placed in the mouth between the cheek and gum and left there for a period of time.

The coconut may be left parked in the cheek and gum as long as the user wishes, even well after the nicotine is gone. The coconut packs in the cheek and gum more like tobacco than gum will, so it will more closely resemble chewing tobacco for the user.

The outer liner paper of the pouch of the invention or tube will be similar in feel to a cigarette paper, more so than the plastic used with the nicotine inhaler. The user may momentarily rest the pack or tube of coconut (or other substrate) in the lips prior to parking between the cheek and gum. The handling of the paper and the mouth feel of the paper will remind the user of handling and placing a cigarette (or cigar) in the mouth.

This present invention will provide a novel nicotine replacement therapy product designed to appeal to both smokers and smokeless tobacco users.

These psychological reminders help the user better deal with the habit (psychological aspects) of quitting while they step down their dependence on the nicotine by using fewer pouches throughout the day or keeping the pouches in the mouth for shorter periods of time.

Nicotine to be delivered in a pouch may range from 1 to 60 mg. Preferably, the dosages are up to about 21 mg nicotine per article. The nicotine may be delivered in short periods of a few seconds or minutes or perhaps controlled release over several hours.

Multiple pouches may be used at the same time subject to dose and use guidelines. As is currently practiced, the method for NRT would encompass a step down of nicotine dosage.

A pouch product with similar or same or different flavors, colors or textures that contains no nicotine will be available as well for the user to continue with the habit of chewing the coconut (or other substrate) as long as they wish after they have stepped down off the nicotine.

In an alternative embodiment, the liner may be wrapped around a substrate and configured similar to a conventional cigar or cigarette. The therapeutic agent may be in the liner, the substrate, or combination thereof.

### DETAILED DESCRIPTION OF THE PREFERRED EMBODIMENTS

Completely edible nicotine delivery system that in addition to addressing physical cravings, also satisfies “habit” of chewing for smokeless tobacco users and for smokers. Coconut with nicotine in a pouch is a different nicotine delivery vehicle than is currently offered. The paper texture ultimately used as an outer liner may also be very similar to the paper texture found in cigarettes or cigars, which will to some degree, remind the smoker of the smoking habit. When it is touched by the hands and first placed in the mouth, it may feel a bit like tobacco paper. The user may hold it for a while in their hand before putting in mouth and when put in mouth, may wrest in between lips momentarily.

The present invention will be like a cigarette or cigar before parking it between the cheek and gum where the saliva in the mouth will cause it to dissolve and deliver the nicotine.

Preferably, the liner is dissolvable. However, dissolvable may be rapid, or alternatively, may take place over time from several minutes to several hours.

As the article of the present invention is configured to be placed in the oral cavity, it should be formed of materials that are substantially edible and/or consumable. Thus, the total composition of the article should preferably comprise components that fall within the limits set forth as Generally Recognized as Safe (GRAS).

Different doses of product may be offered and users can “step down” much like they do with other nicotine replacement therapy (NRT) products.

Shape of pouch may be the same as currently used with tobacco pouches or it may be more tubular, similar to or almost exactly like the diameter of a cigarette. The length of the tube could vary, but initial thoughts are that it would be between 1″ and 2″; however it could be shorter or longer.

Depending on the ingredients, the product could appeal to anyone, not just tobacco users. For example, an energy version of an edible product in an edible/dissolvable pouch may appeal to a smokeless tobacco user just as it would a non tobacco user that just wants something else to chew besides gum, toothpicks etc or perhaps both these users are just after the energy burst.

Although colors may be added, “white” versions of coconut in the article may be more appealing to the user than darker versions or leaf, tobacco or other base ingredients. Being white would make it less noticeable in the mouth and juice could be clear or milky looking versus brown or black. Also, a coconut product is completely edible so if in public place like church or work or restaurant and don't have opportunity to discard non edible pouch and or non edible ingredients like tobacco or leaves, the entire coconut product in a dissolvable/edible pouch could be eaten.

The article may comprise a therapeutic agent which may be, but would not be limited to, pharmaceutical, nutritional supplements, and the like.

The therapeutic agent may be delivered in immediate release, controlled release, delayed release, sustained release, or combinations thereof.

The article may also comprise a nicotine salt placed on the interior. Thus, when the liner dissolves, the nicotine salt remains and provides the NRT desired.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. An article for delivering a product to the oral cavity comprising:
(a) at least one dissolvable material used as a liner, said liner forming a pouch that completely encloses an inner cavity;
(b) a substrate placed within said inner cavity.

2. The article of claim 1 wherein said pouch comprises at least two dissolvable materials that have different rates of dissolution in the oral cavity.

3. The article of claim 1 wherein said article comprises nicotine suitable for nicotine replacement therapy.

4. The article of claim 1 wherein said substrate comprises nicotine suitable for nicotine replacement therapy.

5. The article of claim 1 wherein said liner comprises nicotine suitable for nicotine replacement therapy.

6. The article of claim 1 wherein said substrate is an edible substance.

7. The article of claim 1 wherein said liner is an edible substance.

8. The article of claim 1 wherein said substrate comprises coconut.

9. The article of claim 1 wherein said substrate comprises components such as flavors, herbals, pharmaceuticals, nutritional supplements and the like incorporated into said article.

10. The article of claim 1 wherein said liner comprises components such as flavors, herbals, pharmaceuticals, nutritional supplements and the like incorporated into said article.

11. The article of claim 1 wherein said liner and substrate comprises components such as flavors, herbals, pharmaceuticals, nutritional supplements and the like incorporated into said article.

12. The article of claim 1 wherein said substrate comprises components such as pharmaceuticals,
nutritional supplements and the like incorporated into the lining of the pouch, one or more liners, the inner substrate, or combinations thereof.

13. A method for providing nicotine replacement therapy comprising the steps of:
(a) producing articles according to claim 1 that incorporate different amounts of nicotine;
(b) administering to a person said articles with nicotine content decreasing over time.

14. A method for providing therapy comprising the steps of:
(a) producing articles according to claim 1 that incorporate therapeutic agents;
(b) administering to a person said articles containing said therapeutic agent.
